(19) United States
(12) Patent Application Publication
(10) Pub. No.: US 2024/0358529 A1
(43) Pub. Date: Oct. 31, 2024

(54) FOOT CONTROLLER SYSTEMS FOR PROSTHETIC ARMS, THEIR METHODS OF PRODUCTION AND USE

(71) Applicant: The Trustees of the California State University, Long Beach, CA (US)

(72) Inventors: Peter L. Bishay, North Hills, CA (US); Jack Wilgus, Newbury Park, CA (US)

(73) Assignee: The Trustees of the California State University, Long Beach, CA (US)

(21) Appl. No.: 18/648,726

(22) Filed: Apr. 29, 2024

Related U.S. Application Data
(60) Provisional application No. 63/462,989, filed on Apr. 29, 2023.

(51) Int. Cl. A61F 2/70; A61F 2/58

(52) U.S. Cl. CPC A61F 2/70; A61F 2/585; A61F 2/586; A61F 2002/704; A61F 2002/705; A61F 2002/707

(57) ABSTRACT

Prosthetic arm controller systems, as disclosed and discussed herein, include: a first controller unit that is placed or located inside a shoe or a sock of a user or that is integrated into a foot sleeve that slides on to the foot of a user, wherein the first controller unit comprises at least two interactive buttons that the user can engage, a second controller unit that is removably clipped or otherwise removably attached to the side or top of the shoe or the sock, wherein the second controller unit comprises at least one microcontroller with at least one integrated gyroscope, at least one accelerometer, and an onboard wireless protocol antenna that wirelessly communicates at least one command to a prosthetic arm from the first controller unit or the second controller unit, and wherein the at least one command comprises at least one grip command, at least one rotate command, at least one bend command, or a combination thereof. In some embodiments, the first controller unit and the second controller unit may be connected through a wired connection.

This United States Utility Application claims priority to U.S. Provisional Application Ser. No. 63/462,989 entitled “Foot Controller for Prosthetic Arms, Their Methods of Production and Use” filed on Apr. 29, 2023, which is commonly owned and incorporated herein in its entirety by reference.

### FIELD OF THE SUBJECT MATTER

The field of the subject matter is foot controller systems for prosthetic arms, their methods of production and use.

### DETAILED DESCRIPTION

Controlling prosthetic arms in an easy, effective, and noninvasive way is one of the most challenging tasks. Surface electromyography (sEMG) is the most used approach for prostheses control. Geethanjali [1] presented a state-of-the-art review of myoelectric control of prosthetic hands [2]. sEMG requires some capable muscles to exist in the residual limb to strain the myoelectric sensors. Prosthetic users reported muscle fatigue and unreliability when using myoelectric sensors. Some pattern recognition approaches were proposed to reduce direct control by prosthetic users. However, the main issue of sEMG pattern recognition-based systems is that they rely on the repeatable matching of the produced sEMG patterns during prosthesis manipulation to those used for system training. These patterns tend to significantly change due to environmental factors such as sweat or electrode shift, as well as fatigue, load, limb position, or simply due to the user's change of focus [3-5]. Another drawback of these solutions is their limited ability to successfully cope with simultaneous motions, which makes them still not fully intuitive and somewhat cognitively demanding [6].

Searching for novel and innovative approaches to control upper limb prostheses, alternative control methods have been proposed recently, such as electroencephalography (EEG), or brain wave control [7-9], and voice control [2,9,10]. In general, EEG needs considerable mental concentration to give reliable results and is affected by distractions and the amount of hair in the scalp. Voice control is very reliable but requires the users to talk to their prostheses, which might not always be desirable. In 2020, Hazubski et al. [11] presented a proof-of-concept study on a new approach to control a prosthesis, an exoskeleton, or an end effector visually using augmented reality glasses. In 2022, Nagaraja et al. [12] proposed a breathing-powered system for body-powered prostheses. Sonomyography (SMG) [13-16], or ultrasound-based sensing/imaging, has also been used to control prostheses.

Using the foot to control prosthetic arm motions has been proposed by multiple research groups, but most designs relied on identifying foot postures using sEMG. Most of these designs are intended to supplement arm sEMG, since the residual limb might lack enough muscle to control all degrees of freedom in the prosthesis. For example, Lyons and Joshi [17,18] used sEMG sensors placed on the lower leg and mapped the degrees of freedom of the leg to those of the arm, to enable noninvasive control of prosthetic elbow, wrist, and hand movements with minimal training. This was based on a case study that showed intuitive mapping between the human hand and foot movements [19]. Maragliulo et al. [20] used sEMG foot band as a hands-free wearable human machine interface (HMI) that can classify five foot gestures. They also added a locking/unlocking mechanism controlled by one of the gestures to eliminate undesired gesture classification during general leg movements (walking, jumping, climbing the stairs, etc.). Lee et al. [21] used a wearable fabric sensor on the lower leg to map foot postures to prosthetic hand postures. Their approach relied on convolutional neural networks (CNN) to classify eight leg postures based on pattern recognition. DEKA arm [22,23] included a foot controller that has force sensitive resistors (FSRs) soldered onto foot pads worn inside the shoes, in addition to inertial measurement units (IMU) mounted on a clip that attaches to the top of the shoe. The IMU utilizes gyroscopes and micro-electromechanical systems (MEMS) accelerometers to sense small movements of the foot/ankle.

Nowadays there are wide variety of prosthetic arm designs in literature, the market, and CAD design websites, with different shapes, sizes, degrees of freedoms, actuators, and materials. However, only limited options are available for controlling such prostheses. The market is mainly dominated by myoelectric prostheses, which require some capable muscles to exist in the residual limb to strain the myoelectric sensors. Prosthetic users reported muscle fatigue and unreliability when using myoelectric sensors. Alternative control systems were also proposed, such as electroencephalography (EEG) or brainwave control headsets, and voice control systems. In general, EEG needs considerable mental concentration to give reliable results, and is affected by distractions, and amount of hair in the scalp. Voice control is very reliable but requires the users to talk to their prostheses, which might not always be desirable. Utilizing the leg or the foot to control the motion of a prosthetic arm was proposed. However, most designs included inertial measurement units (IMUs) placed on the leg to sense the foot posture that can be mapped to hand posture. DEKA arm developers also realized a foot controller that relies on force sensors placed under the shoe insole.

### SUMMARY OF THE SUBJECT MATTER

A prosthetic arm controller system, as disclosed and discussed herein, includes: a first controller unit that is placed or located inside a shoe or a sock of a user, wherein the first controller unit comprises at least two interactive buttons that the user can engage, a second controller unit that is removably clipped or otherwise removably attached to the side or top of the shoe or the sock, wherein the second controller unit comprises at least one microcontroller with at least one integrated gyroscope, at least one accelerometer, and an onboard wireless protocol antenna that wirelessly communicates at least one command to a prosthetic arm from the first controller unit or the second controller unit, and wherein the at least one command comprises at least one grip command, at least one rotate command, at least one bend command, or a combination thereof. In some embodiments, the first controller unit and the second controller unit may be connected through a wired connection.

In another embodiment, a prosthetic arm controller system, as disclosed and discussed herein, includes: a first controller unit that is incorporated in a foot sleeve, wherein the first controller unit comprises at least two interactive buttons that a user can engage, a second controller unit that is removably clipped or otherwise removably attached to the foot sleeve, wherein the second controller unit comprises at least one microcontroller with at least one integrated gyroscope, at least one accelerometer, and an onboard wireless protocol antenna that wirelessly communicates at least one command to a prosthetic arm from the first controller unit or the second controller unit, and wherein the at least one command comprises at least one grip command, at least one rotate command, at least one bend command, or a combination thereof. In some embodiments, the first controller unit and the second controller unit may be connected through a wired connection.

### BRIEF DESCRIPTION OF THE FIGURES AND TABLES

FIG. 1 shows a contemplated prosthetic arm controller system 100 that comprises an insole controller 110 and a sensor-controller unit (SCU) 150.

FIG. 2 shows a foot sleeve 200 is worn by a user 210, wherein the foot sleeve incorporates and supports the necessary controls (220, 250).

FIG. 3 shows the system architecture diagram of the data transfer protocol for a contemplated foot controller system.

FIG. 4 shows the overall architecture diagram of a contemplated foot controller system and the prosthetic arm it controls.

FIG. 5 shows a corresponding illustration 500 of the grips/gestures 510, a hand demonstration 520, and the corresponding toe clicks or foot control 530, for two possible control approaches.

FIG. 6 shows the full CAD assembly of a contemplated prosthetic arm or Infinity arm 600. The forearm 610 houses the wrist actuation mechanism 615, which is connected to the palm 620. The palm 620 includes lightweight digital/micro servomotors 625 that actuate the fingers 630 via tendons (not shown).

A contemplated wrist actuation system 700 is shown in FIG. 7.

Exploded 800 and assembled 810 views of the palm structure 820 and fingers 830 are shown in FIG. 8A (exploded) and FIG. 8B (assembled).

The arm's architecture diagram 900 is presented in FIG. 9, following a contemplated control approach 1.

FIGS. 10A and 10B show, respectively, a contemplated foot controller system 1010 with the insole controller 1020 ready to be placed inside the shoe 1030 and the SCU 1040 clipped to the side of the shoe 1030.

The system includes a training platform similar to the front part of the foot controller insole 1100, as shown in FIG. 11, with two push buttons (not shown) under the paddles 1110.

Phase 1 is a sequence of FIGS. 1210 that represent tasks the trainee should do by pressing the paddles with their toes, as shown in the examples in the first column in

FIG. 12. Phase 2 does not show the trainee any pictures of toes as in phase 1 but shows pictures 1220 of a human hand doing one grip per slide, as shown in the examples in the second column in FIG. 12. Phase 3 gets even more challenging by showing the trainee pictures of real-life objects, as shown in the examples in the third column 1230 in FIG. 12.

Table 1 shows an example of foot control actions and their corresponding prosthetic arm reactions, for two possible and contemplated control approaches. Since these reactions are controlled via an editable microcontroller code, any different reactions can be implemented.

Table 2 is a bill of material that includes all components used in the proof-of-concept models of Infinity foot controller and arm. The total weight of the arm without a socket is 0.72 kg (1.6 lbs).

### DETAILED DESCRIPTION

As mentioned, contemplated embodiments include a foot controller system that is used to control a trans-radial (below-the-elbow) prosthetic arm. The system includes an insole or a foot sleeve controller (a first controller unit) and a sensor-controller unit (SCU), which is also referred to as a second controller unit. Commands from two push buttons integrated in the insole or foot sleeve underneath the toes, and from the gyroscopes and accelerometers in the SCU, are transmitted wirelessly to the prosthetic arm to apply different grips and rotate or bend the prosthetic hand.

Specifically, a prosthetic arm controller system, as disclosed and discussed herein, includes: a first controller unit that is placed or located inside a shoe or a sock of a user, wherein the first controller unit comprises at least two interactive buttons that the user can engage, a second controller unit that is removably clipped or otherwise removably attached to the side or top of the shoe or the sock, wherein the second controller unit comprises at least one microcontroller with at least one integrated gyroscope, at least one accelerometer, and an onboard wireless protocol antenna that wirelessly communicates at least one command to a prosthetic arm from the first controller unit or the second controller unit, and wherein the at least one command comprises at least one grip command, at least one rotate command, at least one bend command, or a combination thereof. In some embodiments, the first controller unit and the second controller unit may be connected through a wired connection.

In another embodiment, a prosthetic arm controller system, as disclosed and discussed herein, includes: a first controller unit that is incorporated in a foot sleeve, wherein the first controller unit comprises at least two interactive buttons that a user can engage, a second controller unit that is removably clipped or otherwise removably attached to the foot sleeve, wherein the second controller unit comprises at least one microcontroller with at least one integrated gyroscope, at least one accelerometer, and an onboard wireless protocol antenna that wirelessly communicates at least one command to a prosthetic arm from the first controller unit or the second controller unit, and wherein the at least one command comprises at least one grip command, at least one rotate command, at least one bend command, or a combination thereof. In some embodiments, the first controller unit and the second controller unit may be connected through a wired connection.

It should be understood that the phrase “first controller unit” includes the “insole controller”, “insole controller unit”, “forefoot sleeve controller”, “forefoot sleeve controller unit”, “foot sleeve controller”, “foot sleeve controller unit”, “sleeve controller”, or “sleeve controller unit”, as used herein and those phrases can be used interchangeably to mean the same thing in this disclosure. It should also be understood that “second controller unit” includes the “sensor-controller unit” or “SCU” and those phrases can be used interchangeably to mean the same thing in this disclosure. It should be understood that the phrase “prosthetic arm controller system” includes the “Infinity Foot Controller” or “Infinity System”, as used herein and those phrases can be used interchangeably to mean the same thing in this disclosure.

As used herein, an onboard wireless protocol antenna that wirelessly communicates at least one command to a prosthetic arm from the first controller unit or the second controller unit means a Bluetooth antenna. In other embodiments, the onboard wireless protocol antenna may be another protocol other than Bluetooth.

As mentioned, in some embodiments, the first controller unit and the second controller unit may be connected through a wired connection. In other contemplated embodiments, the first controller unit and the second controller unit are connected through a wired connection.

Contemplated embodiments include the design of the “Infinity Foot Controller” system to control a trans-radial (below-the-elbow) prosthetic arm. Contemplated embodiments include an insole controller to be placed inside a shoe or a sock or a foot sleeve controller unit that is incorporated into a foot sleeve that is worn on a bare foot and a sensor-controller unit (SCU) to be clipped to the side or top of the shoe or the sock, or to the top of the foot sleeve, to control the prosthetic arm, including finger deformation, rotation and bending of the wrist, and/or bending at the elbow. Commands from two push buttons integrated in the insole or foot sleeve underneath the toes, and/or from the gyroscope and accelerometers in the SCU, are transmitted wirelessly to the prosthetic arm to apply different grips and rotate or bend the prosthetic hand. The push buttons also provide audible feedback and haptic feedback to the user's toes. In order to test and demonstrate the foot controller, a trans-radial 3D-printed arm, called the “Infinity Arm”, was designed with a capability to grip lightweight everyday objects, as well as bend and rotate the wrist. Preliminary tests demonstrated the ability of humans to utilize the controller effectively after minimal training.

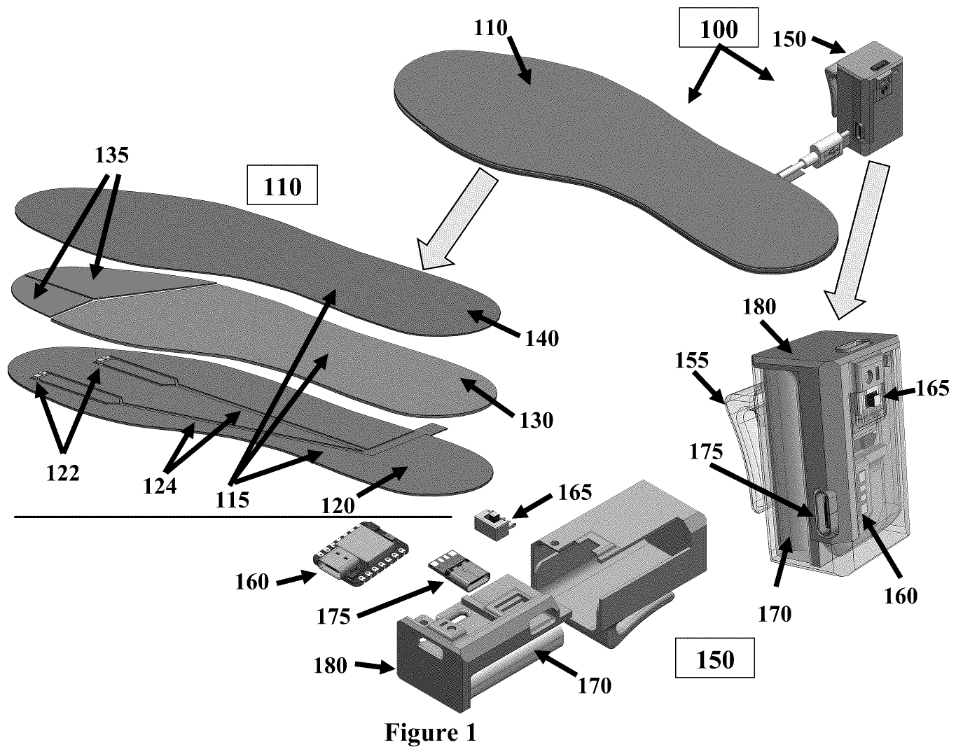

A contemplated prosthetic arm controller system 100 is shown in FIG. 1. It comprises an insole controller 110 and a sensor-controller unit (SCU) 150. The insole controller is a sandwich structure of three layers 115. The bottom layer 120 is 3D-printed from thermoplastic polyurethane (TPU) and has two controlling push buttons 122 located underneath the toes (not shown), and wiring channels 124 that allow for proper wire management. The middle layer 130, which is a silicone sheet in this embodiment, includes polylactic acid (PLA) paddles 135 placed above the push buttons 122 to increase the surface area on which the user applies a force with their toes. The top layer 140 is fabric for added comfort. A silicon molding adhesive (not shown) in the middle layer 130 is used to bond the top and bottom layers together once all components and wires are assembled properly. The silicon molding adhesive also serves as a shock absorber and an insulator for all electronic components. The shape of all layers is modeled after traditional insoles, sized 5-13.

The SCU 150 housing is 3D-printed from PLA and is clipped to the shoe (not shown) via a universal clip 155 integrated in the outer shell to allow for secure mounting. A contemplated SCU 150 includes housings for a Seeed Studio XIAO nRF52840 Sense microcontroller 160 with an internal with an internal Inertial Measurement Unit (IMU) and onboard wireless protocol and/or Bluetooth antenna, a power slide (on/off) switch 165, a rechargeable 3.7V 400 mAh LiPo battery 170, and a USB-C female plug 175, all mounted on a PLA mounting structure 180. The battery charging management capability of the microcontroller indicates the charge level with an LED and ensures that the Li-Po battery would not be overcharged. The microcontroller receives signals from the push buttons and the IMU's sensors and transmits these signals to the prosthetic arm to grip with the fingers, to bend or rotate the wrist, or bend at the elbow joint. The IMU's gyroscope measures the foot rotation around the yaw-axis (dorsiflexion and plantarflexion) and around the pitch-axis (inversion and eversion) to, respectively, control bending (wrist extension and flexion) and rotation (supination and pronation) of the prosthetic arm's wrist. The rotation around the roll-axis (internal and external rotations) may also be used to control either wrist deviation (abduction and adduction) or elbow rotation if these degrees of freedom are added to additional contemplated embodiments of the arm.

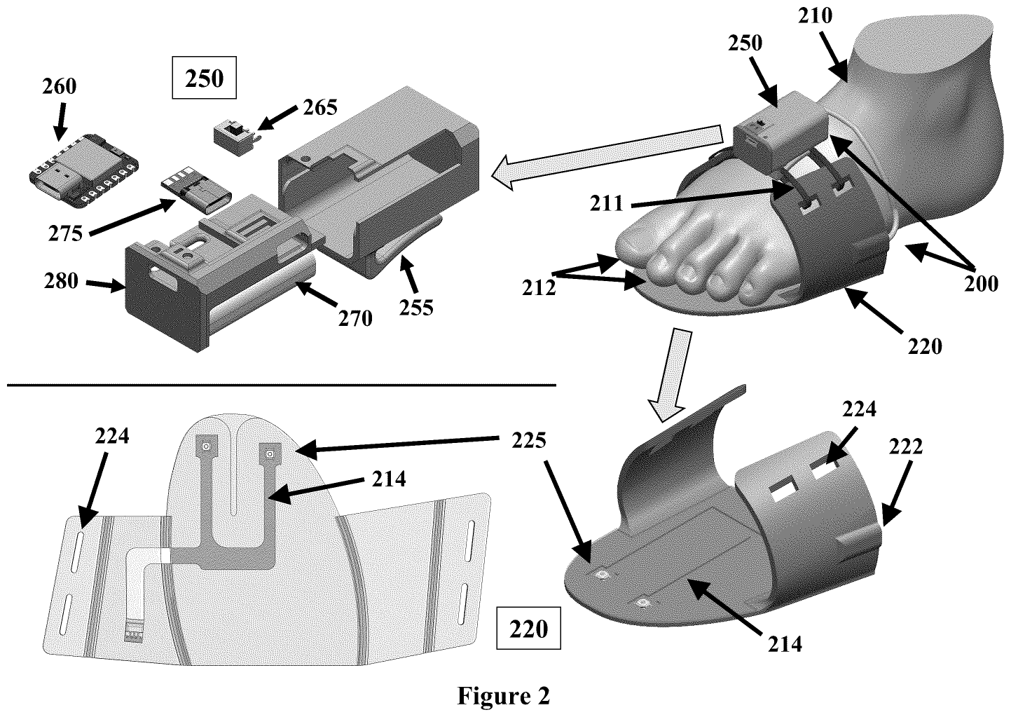

In another contemplated embodiment shown in FIG. 2, wherein a foot sleeve controller 200 is worn by a user 210 and wherein the foot sleeve incorporates and supports the necessary controls (220, 250). It should be understood that this foot sleeve controller design 200 could be incorporated into a sandal or “flip flop” design as well (not shown). This embodiment comprises a forefoot or foot sleeve controller 220 and a sensor-controller unit (SCU) 250, which is identical to the SCU 150 in the first contemplated embodiment. The sleeve controller 220 is 3D-printed of/with TPU, a USB-C port 222, and has two controlling push buttons 225 located underneath the PLA paddles (not shown), similar to the paddles in the insole of the first contemplated embodiment, which are located below the toes 212. The sleeve has grooves for the push buttons and wire channels 214 and openings for laces or VELCRO tape 224. A layer of fabric (not shown) covers the inner surface of the sleeve to protect the electric wires and add comfort. The USB-C port enables the connection to the SCU 250. The SCU 250 housing is 3D-printed from PLA and is clipped to sleeve's laces or Velcro tape 211 via a universal clip 255 integrated in the outer shell to allow for secure mounting. A contemplated SCU 250 includes housings for a Seeed Studio XIAO nRF52840 Sense microcontroller 260 with an internal IMU and onboard wireless protocol and/or Bluetooth antenna, a power slide (on/off) switch 265, a rechargeable 3.7V 400 mAh LiPo battery 270, and a USB-C female plug 275, all mounted on a PLA mounting structure 280.

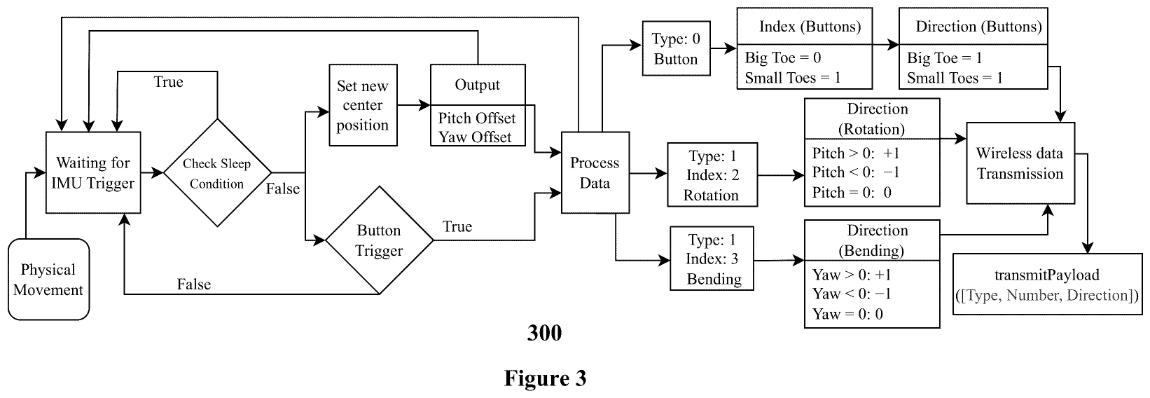

Data transfer between the foot control and the arm can be accomplished in a number of different and contemplated ways. For example, it can follow a custom protocol that relies on a “payload” made of three integers in a 1×3 array: [Type Index Direction]. The “Type” integer is either 0 for buttons, or 1 for a gyroscope axis shift. The “Index” integer is 0 for the big toe button press, 1 for the smaller toes button press, 2 for rotation (pitch gyroscope axis shift), or 3 for bending (yaw gyroscope axis shift). Finally, the “Direction” integer is used only for rotation and bending and takes a value of +1 or −1 to indicate the direction of rotation or bending, or a value of 0 if the old position is the same as the new position. An example of this payload would be [0 1 1], where the first value (0) indicates a button pressed, and the second value (1) represents the small toes button. The last value is ignored in this case of a button press. Another example is [1 2 −1] which indicates an axis shift trigger (1), and this triggered axis is the pitch axis (2), and the rotation direction is negative (−1), which is to the right in this case. The system architecture diagram 300 of this custom data transfer protocol for the foot controller system (not shown) is in FIG. 3, which is referred to as a contemplated control approach 1.

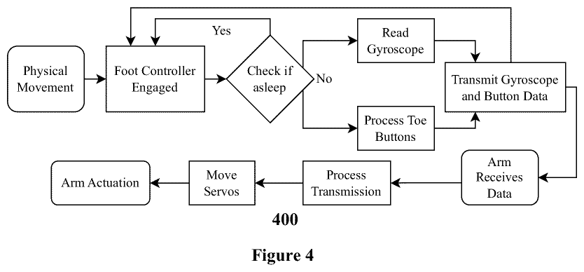

Once any physical movement happens in the foot, the IMU will be triggered, and the code checks if the Sleep condition is met. If the SCU is not in the Sleep mode, the pitch and yaw offsets will be calculated after setting a new center position for the axes. Also, the two buttons will be checked for a trigger. The measured data (buttons pressed or axes shifted) are then processed to assign “Type”, “Index”, and “Direction” for the payload array to be transmitted wirelessly. The overall architecture diagram 400 of the foot controller system (not shown) with the arm (not shown) is in FIG. 4. Physical movement of the foot is captured if the foot controller is engaged and is not in the Sleep mode. The data of gyroscope axes shifts and button presses are collected and transmitted wirelessly to the arm. Once the arm's microcontroller receives the payload data, it processes it and accordingly moves the servomotors to actuate the arm.

The microcontroller code runs a series of loops to move the desired motor in the desired direction. These loops control the speed, check if the movement is still being engaged, and include a safety stop to prevent over-actuation. In each iteration of the loop, a check is made to determine if the foot controller's gyroscope action is still being desired. Once the foot stops, the loop ends, and the wrist rotation or bending stops. This happens almost instantaneously since this loop iterates every 20 ms, and in each iteration, the arm servomotor rotates 1°. Rotational speed can be adjusted in two ways: (1) by controlling the loop refresh rate (increasing or decreasing this 20 ms to make the loop iterate faster or slower, respectively), or (2) by controlling the number of degrees the arm servomotor rotates in each loop iteration. One degree per loop used in the developed code allowed for a high resolution of rotation or bending without any jitter. The safety stop is only engaged when the degrees of rotation of each servomotor reach the maximum amount that was set beforehand during the initial calibration.

Another possible approach (contemplated control approach 2) would be to dedicate the big toe button to gradual finger closing, and the lesser toes button to gradual finger opening. Pressing and holding any of the two buttons will continuously close or open a set of fingers. Releasing the button at any time would stop finger actuation at the current configuration. This approach allows the users to control the amount of force applied on any object to be gripped, as finger deformation is happening gradually based on the duration of the button hold. Changing the set of fingers to be actuated can be done by clicking both buttons simultaneously. Simultaneous clicks would cycle through sets of fingers to be actuated. For example, the cycle can include: set 1: thumb and index (for pinching), set 2: thumb, index, and middle (for tripod grip), set 3: all fingers (for power grip), set 4: all fingers except the index (for point gesture), set 5: all fingers except the thumb (for thumbs-up gesture), and set 6: all fingers except index and middle (for peace sign gesture). Sets assigned in the cycle can be customized for each user.

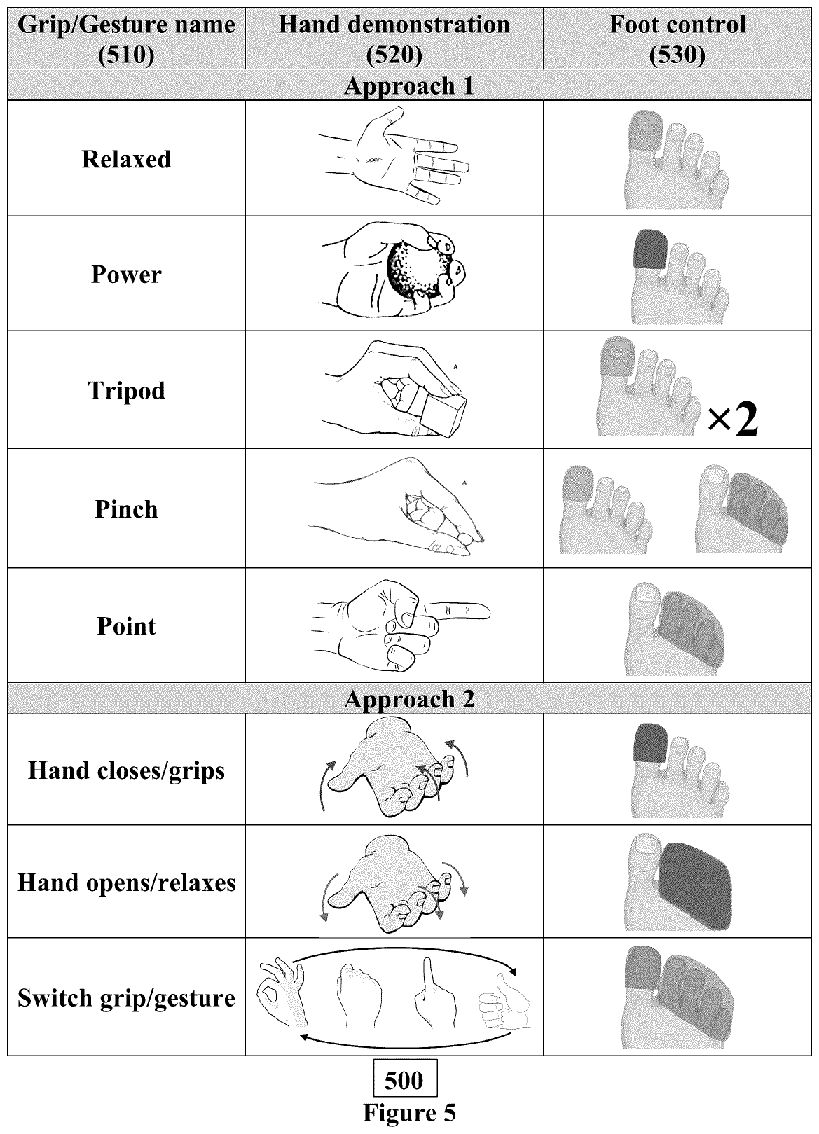

Table 1 shows foot control actions and their corresponding prosthetic arm reactions following the two mentioned control approaches. Since these reactions are controlled via an editable microcontroller code, any different reactions can be implemented. FIG. 5 shows a corresponding illustration 500 of the grips/gestures 510, a hand demonstration 520, and the corresponding toe clicks or foot control 530, for both presented control approaches.

Since walking can result in unintended pressing on the push buttons, the foot controller is intended to be used only while sitting or standing. A walking detection system was developed. This system uses both the accelerometers and gyroscope of the SCU to detect two conditions: (1) if motion above the set thresholds is happening in all three axes of the gyroscope, and (2) if the current state of the foot controller is a “rest” state. The second condition is checked only if the first condition passes its check. If either of these conditions fail their check, the controller will consider the user walking and not ready to use the controller as intended. Once both conditions pass, the controller will set a new rest orientation to be used for subsequent checks. Finally, if the controller detects rapid movement when either of the conditions has failed the check, it pauses checking the IMU signals for 2.5 s and then resumes its normal operation to determine if the user is ready to use the controller to control the prosthetic hand. When the system enters the “movement” state (when walking), the arm would lock its grip until the accelerometers detect the user sitting or standing (going to the “rest” state), and then the signal transmission will be resumed.

While contemplated embodiments center around the foot controller system, it is important to put it into context by showing what the foot controller system controls: the prosthetic arm. An overview of existing 3D-printed upper limb prostheses, including the benefits and drawbacks of 58 designs, was presented by Ten Kate et al. [24]. Andrés et al. [25] made a comparison between tendon and linkage prosthetic transmission systems. They concluded that the tendon-driven model achieved a greater quantity of successful grasps compared to the linkage-driven model. Tendon-driven hands are dominant because of the fewer number of parts to be printed, the easier assembly for a nonexpert user, and the advantages in pursuit of lightweight devices. To test and demonstrate the developed foot controller, a 3D-printed below-the-elbow prosthetic arm has been designed. This arm, named “Infinity” arm, is meant to be used for gripping lightweight objects, such as a cell phone, a cup, a piece of fruit, a book, etc., or for making hand gestures, such as pointing, thumbs-up, etc. Due to the lightweight limited-torque servomotors used and the material properties of the 3D-printed plastic constructing it, this arm is unsuitable for sports, carrying heavy objects, and performing harsh tasks. Infinity arm has only the degrees of freedom that the foot controller is targeting.

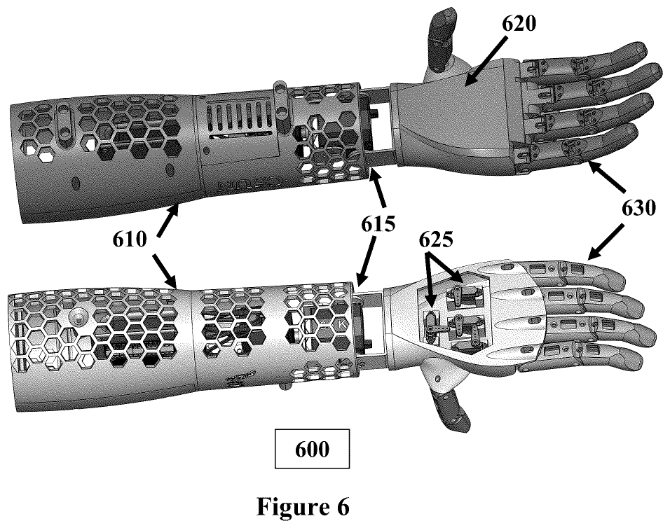

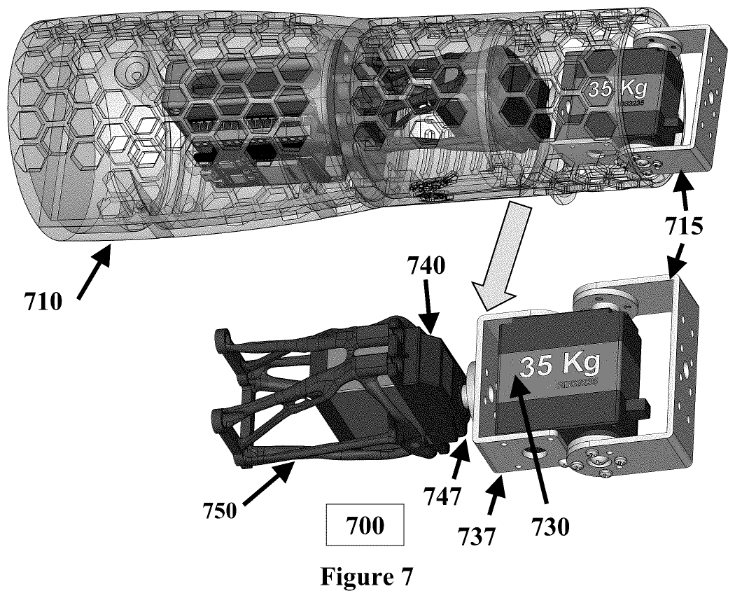

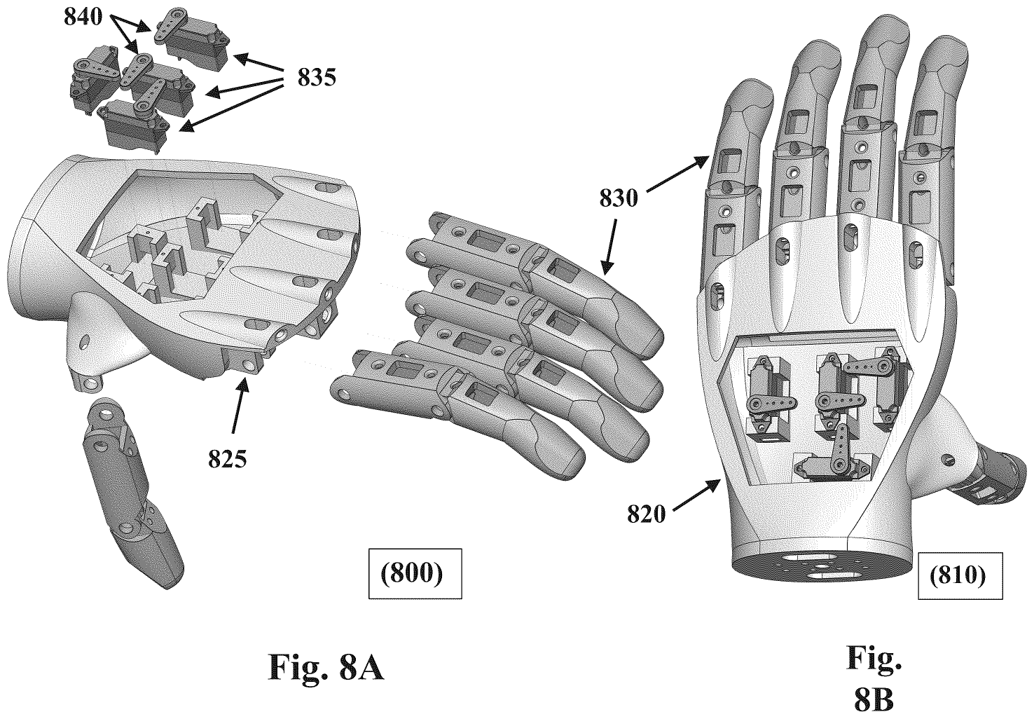

FIG. 6 shows the full CAD assembly of a contemplated prosthetic arm or Infinity arm 600. The forearm 610 houses the wrist actuation mechanism 615, which is connected to the palm 620. The palm 620 includes lightweight digital/micro servomotors 625 that actuate the fingers 630 via tendons (not shown).

Both the academic and industrial research communities have tended to place more focus on hand/gripper development than on wrist actuation systems [26]. Montagnani et al. [27] showed that increased dexterity in wrist prostheses may contribute more to manipulation capacity than a highly dexterous terminal device with limited wrist capability. Bajaj et al. [26], in 2019, presented a state-of-the-art review on 1D, 2D, and 3D prosthetic and robotic wrist actuation designs. In 2022, Fan et al. [28] presented another review of artificial prosthetic and robotic wrists evaluating their mobility, stability, output capability, load capacity, and flexibility compared to the human hand.

A contemplated wrist actuation system 700 is shown in FIG. 7. This system, which is attached to the forearm structure 710 and palm (not shown) through a U-shaped aluminum holder 715, connected to RDS3235SG 35 kg dual bearing coreless servomotor 730 (stall torque: 30 kg·cm at 7.4 V; max. speed: 0.11 s/60°; weight: 60 g; size: 40×20×40 mm). Torque from the servomotor rotates the aluminum holder to bend the hand assembly by up to 35° in both directions. Rotation is achieved using a DS3235 35 Kg coreless servomotor 740 (stall torque: 30 kg·cm at 7.4 V; max. speed: 0.11 s/60°; weight: 60 g; size: 40×20×38.5 mm) placed below the central structure within the forearm. The wrist rotation servomotor 740 can rotate the wrist bending servomotor 730 through the circle holder 747 and the U-shaped aluminum mount 737 that connect the two servomotors. The wrist rotation servomotor 740 is secured to a 3D-printed PLA servomotor mount 750, which is fixed to the forearm structure 710. This design allows rotation of up to 60° in both directions.

Inspired by the human hand and the previously designed prosthetic arms [29], the shape and size of Infinity's palm design resembles that of an average male hand. Exploded 800 and assembled 810 views of the palm structure 820 and fingers 830 are shown in FIG. 8A (exploded) and FIG. 8B (assembled). The palm structure 820 has attachment points 825 for the fingers 830 that are secured using pins (not shown). Only four integrated KST X06V micro servomotors 835 (stall torque: 1.8 kg·cm at 8.4 V; max. speed: 0.07 s/60°; weight: 6 g; size: 20×7×16.6 mm) are used to actuate the five fingers 830, because the pinky and ring fingers are connected to one servomotor and actuate together. These two fingers deform similarly in every grip/gesture of this hand. Finger flexion is produced by the torque originating from the servomotor which rotates a servo horn 840 mounted on top of the motor. The actuation tendons (not shown) are tied to the servo horns 840 and are routed through designated pathways inside the fingers 830 where they are attached to the fingertips. Thus, as each servomotor 835 rotates up to 160°, it actuates the corresponding finger. Extension of each finger is achieved by elastic bands (not shown) that are routed through the finger joints to allow the fingers to return to a relaxed position. Unlike the human biological structure of fingers which have three phalanges, each finger of the proposed design has two main phalanges to maintain simplicity. The distal and proximal phalanges are made as one part with a slight curvature similar to the relaxed posture of human fingers.

With the contemplated first control approach, the thumb and index fingers are actuated in all grips as shown in FIG. 5. The middle, ring, and pinky are only actuated in specific grips/gestures. The order in which these fingers actuate is (1) thumb, (2) index, (3) middle, (4) ring and pinky combined. The thumb is actuated first to move the object closer to the palm, then the index is next to grab the object. The other fingers are there to support the object in any way they can. The speed the fingers are being actuated is controlled in a way similar to the bending and rotation system. The only difference is that the fingers' full range of motion is set on a scale of 0 to 1, where 0 represents 0% and 1 represents 100%. The speed can be controlled by adjusting the percentage value to get to the end point assigned to the set grip inside the loop. A 0.05 (5%) step per loop was found reasonable. This means that a full finger closure can happen in 20 loops (to reach 1 or 100%). With a duration of 20 ms per loop, each finger closure would take 400 ms (0.4 s) to be achieved. Final finger position is preset for each grip during calibration. With the contemplated second control approach, also shown in FIG. 5, the fingers of the selected grip keep deforming as long as the big toe is pressing on the big toe push button. The fingers stop once the toe is no longer pressing on the button or once the maximum allowable finger deformation is reached, corresponding to the mechanical limit of the finger actuating servomotor.

The arm's architecture diagram 900 is presented in FIG. 9. Once the arm is powered, it centers all servomotors, and waits for any data transfer. Once data is received as a payload from the foot controller, button presses are used to actuate the fingers according to the identified grip and bending/rotation data are used to actuate the wrist bending and rotation servomotors in the desired direction. Specifically for the big toe button presses, if control approach 1 is used, the code identifies if it is a single, double, or a long click, to engage the correct grip.

All structural components were 3D-printed, and servomotors were secured to their housings. Tendons were routed through the fingers and tied to the servo horns on the servomotors, finger digits were assembled to the palm structure using pins, and all electric wires were connected to the main circuit board. Table 2 is a bill of material that includes all components used in the proof-of-concept models of Infinity foot controller and arm. The total weight of the arm without a socket is 0.72 kg (1.6 lbs).

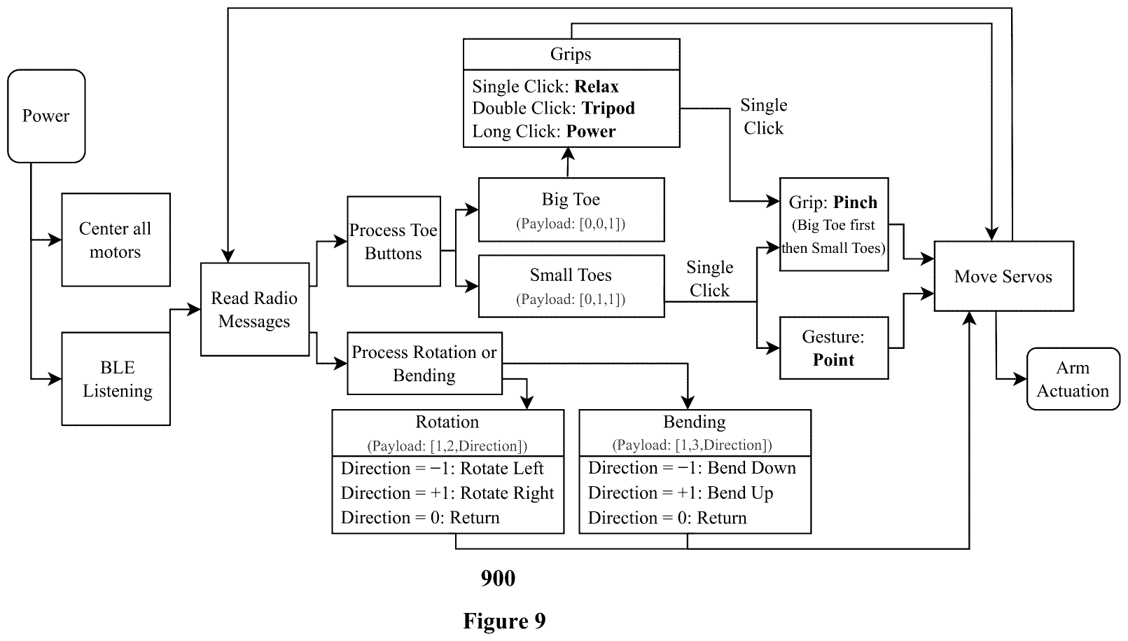

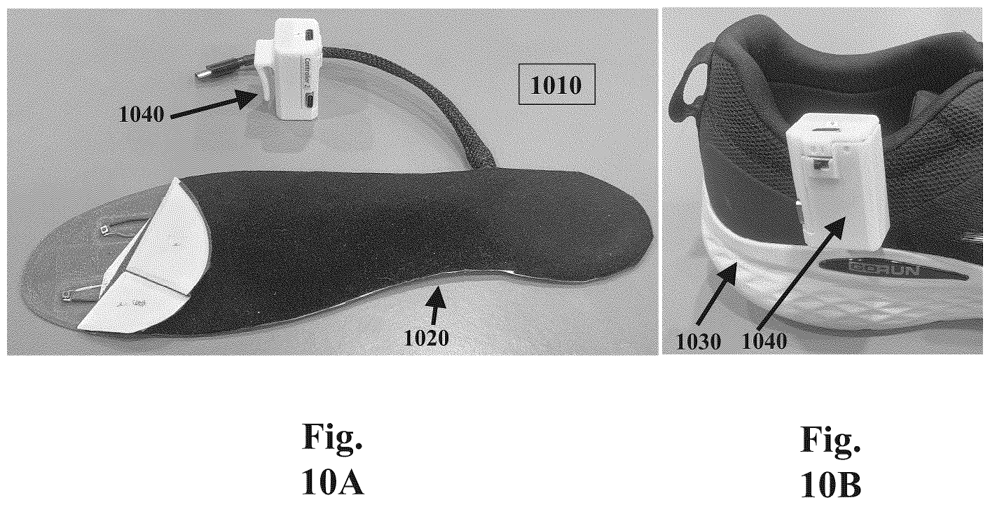

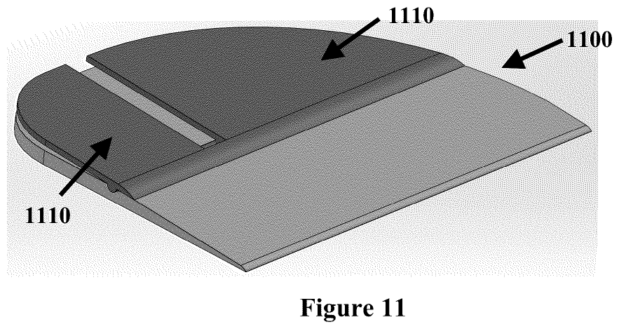

The TPU layer and the PLA paddles of the foot controller were 3D-printed, then the push buttons were secured in their assigned housings in the TPU layer, and the electric wires were routed through their channels. The silicon and fabric layers were then bonded to complete the insole or sleeve controller. The SCU housing was also 3D-printed, and all its electronic components were secured in their assigned positions. FIG. 10A and 10B shows a contemplated foot controller system 1010 with the insole controller 1020 ready to be placed inside the shoe 1030 and the SCU 1040 clipped to the side of the shoe 1030.

A proof-of-concept Infinity Arm model was built and tested performing the five finger grips/gestures it was programmed to do as a result of foot control commands as shown earlier in FIG. 5. Rubber fingertips have been added to increase the friction between the fingers and the objects to be gripped. The arm's performance in terms of applying grips matched exactly all foot control signals in all tests.

A contemplated arm system was tested gripping different objects of different weights, shapes, and sizes, such as a cell phone, a wireless computer mouse, an adhesive tape dispenser, a 3D-printed spherical object, a full juice bottle, a pencil, a book, wooden blocks, cups, and plates. The fingers can effectively adapt to the shape of the object it grips. The success of the grip depends on the hand orientation around the object, the extent of contact between the hand and the object, and the material of the object. Speed of finger deformation was adjusted after the initial tests. The high speed that was used initially made it challenging to grip smaller objects since the first finger that touches the object can push it away before the other fingers surround it to grip. Lower speeds of finger deformation applied in sequence proved to improve the chances of successful grips.

Control of upper limb prostheses remained an area that needed a revolution against the traditional myoelectric approach that led to high rates of prostheses rejection. The Infinity Foot Controller system, as disclosed herein, is a compact device that enables the user to simply slide a custom insole in their shoe or sock, or wear a lightweight sleeve on their bare foot, clip a small sensor-controller unit (SCU) to the side or top of the shoe or sock or to the top of the foot sleeve, and perform different tasks using any compatible prosthetic hand equipped with means of wireless communication. Different grips and wrist rotations can be controlled, respectively, using a few clicks on two paddles under the big toe and four lesser toes, along with foot rotations. Infinity's foot controller offers some features that are lacking in previously published foot controllers for prosthetic arms. To demonstrate the system, the 3D-printed “Infinity Arm” was designed with four micro servomotors housed in the palm to actuate the five fingers via tendons. The palm can rotate or bend via a wrist actuation mechanism housed in the forearm. The Infinity Foot Controller utilizes an underutilized human ability to move their toes to perform useful tasks. Testing results confirmed the effectiveness of the developed electronic circuits and microcontroller code, as well as the functionality of the proposed systems.

Contemplated controllers are mainly relying on two switches to perform grasping, but the use of click patterns (single, double, long, and simultaneous clicks) is extending the allowable control signals, with a higher potential for further extension by adding more patterns or more switches at possibly different locations around the toes. sEMG relies on muscle straining in the residual limb, which could not be available in the first place. With push buttons, the proposed controller has a high signal quality, unlike sEMG that struggles from this problem and requires artificial intelligence to help in signal identification.

### Testing and Training System

A training system was prepared to help users master utilizing the foot controller to move the prosthetic arm and perform daily tasks in a short time, even before the physical arm is fitted to them. The system includes a training platform similar to the front part of the foot controller insole 1100, as shown in FIG. 11, with two push buttons (not shown) under the paddles 1110. The platform is connected to an electric circuit that has two LED lights to be placed on a table in front of the trainee during the training sessions. The platform is also connected to an Arduino microcontroller that sends all button presses to a script that is read by a MATLAB code. To start training, the trainee would sit on a chair and place their foot on the testing platform, then watch the screen that shows the commands that the trainee is supposed to perform with their toes on the platform. Each of the two LED lights corresponds to one push button in the training platform. The function of the LED lights is to help the trainee ensure they are clicking the right push buttons, without looking at their feet. Each training session has three phases, following an initial free practice time. The instructions on how the foot controller would activate each grip would first be presented to the trainee. The trainee would also receive a hard copy of this figure in case they need to recall how to activate any grip during the training session.

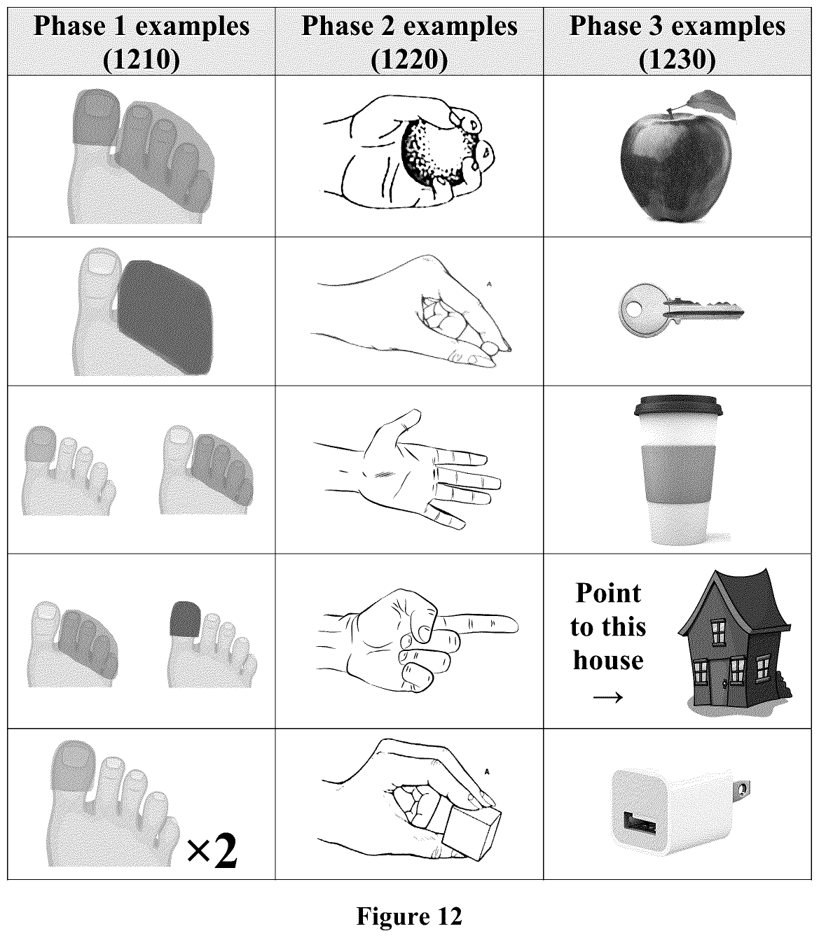

During the initial free practice time, the trainee can try any grip pattern, check that the LED lights are representing the push button presses, and become familiar with the whole system. Phase 1 is a sequence of FIGS. 1210 that represent tasks the trainee should do by pressing the paddles with their toes, as shown in the examples in the first column in FIG. 12. The transparent area on the toes represent single clicks with the big toe or the four lesser toes. The dark areas represent long clicks, and “×2” symbol means repeat two times. If there are two pictures in the slide, as in the third and fourth rows, the commands should be done in sequence from left to right. The MATLAB code compares the received data from the platform to the pre-saved model answers and calculates the score whenever the trainee wants to check their success rate.

Phase 2 does not show the trainee any pictures of toes as in phase 1 but shows pictures 1220 of a human hand doing one grip per slide, as shown in the examples in the second column in FIG. 12. The trainee in this phase would have to translate each hand grip picture in their mind into a toe command, so it is more advanced than phase 1. Phase 3 gets even more challenging by showing the trainee pictures of real-life objects, as shown in the examples in the third column 1230 in FIG. 12. The trainee would have to select a suitable grip that would be successful in gripping the object and translate this into a toe command. Some objects can be gripped using more than one grip. For example, the wall charger block, shown in the last row in the third column, can be gripped using pinch, tripod, or power grips. The MATLAB grading code would consider all possible grips acceptable. However, the apple or the cup would need a power grip to ensure a secure grip. After each of these examples, a slide would ask the trainee to release the object, which would require relax hand posture. The training is customized, so each trainee can repeat any phase multiple times or start from a more advanced level. The code also displays encouraging messages or feedback messages based on score history and mistake type and frequency. Example messages include “Good job, keep it up!” and “Excellent work! You mastered it!” for improved performance, “Keep focusing on your toes and do not rush!” and “Maybe a quick refresher would be useful now” for declining performance or specific repeated mistakes of the same type.

Preliminary testing by the developers of the training system helped select the easiest and most intuitive foot controller action for each hand function. The developers voluntarily consented to try the system just to guide the design choices. The following conclusions were also drawn: (1) in general, a person can reach a very high level of control when using Infinity's foot controller after the first 5-10 min of training, (2) performance in using the foot controller improves quickly with longer training times and more training sessions, (3) pressing buttons with the big toe is the easiest, followed by the lesser toes moving together, since moving an individual lesser toe is way more challenging, (4) receiving feedback (audible and/or haptic) from the push buttons helps improve the user's performance and confidence. A contemplated training system can be extended to include a simulated or a virtual arm that the user can see moving during the training.

After reviewing the contemplated embodiments disclosed herein, it is important now to understand the deficiencies in the DEKA products, especially as applied to this technology space. DEKA Products currently holds two patents and one patent application, including EP 2114316, U.S. Pat. No. 10,499,851, and US Patent Publication 2022/0117759 related to this technology. While it may seem as though this DEKA technology is similar to the contemplated embodiments that were discussed and disclosed herein-they are not. It is important to highlight the differences between Infinity's foot controller and DEKA's foot controller.
DEKA foot control design is intended to be implemented underneath the insole of a shoe whereas a contemplated insole controller is placed on top of the insole or as a replacement of the user's shoe insole. As also disclosed herein, there are contemplated embodiments where the insole can be replaced by a forefoot sleeve, and that can be worn on a bare foot. These embodiments are not taught or suggested by the DEKA products or intellectual property.
Contemplated designs use two push buttons strategically placed underneath the big toe and the four lesser toes. Paddles are placed above these push buttons to increase the surface area of the push buttons for easier control by the user. In addition, the combination of the push buttons and paddles allows for audible feedback to the user as well as the haptic feedback to the user's toes. This allows the user to gain instant feedback of the commands they are sending to the prosthetic arm. On the other hand, DEKA design has only pressure sensors that give no audible or haptic feedback.
In DEKA's foot control system, motions of the prosthetic arm are solely based on differences in force or pressure of the user's foot, whereas contemplated embodiments focus on different commands implemented by the two push buttons under the toes, such as single click, double click, long click, combination clicks, and many other possible commands.
In contemplated embodiments, control of the prosthetic arm gripping actions is localized under the toes which are the body parts that humans can control the most and easiest in their foot. On the other hand, DEKA's control is spread all over the place under the foot, which might be more challenging to use.
DEKA patents state that there may be at least one or more CPUs within the system to translate input data from the sensors to the prosthetic arm. There is no mention of how this CPU is integrated within the system. All what is stated is that the CPU can communicate with the prosthetic arm via wired connection or wirelessly. This application shows the full design of a compact 3D-printed “Sensor-Controller Unit” that includes an IMU and an onboard Bluetooth antenna integrated in a microcontroller, rechargeable battery, and an On/Off power switch. The structure of this unit has been designed to house all these components in the smallest possible footprint that clips to the side or top of the user's shoe, or to the top of the sleeve.

### References

Several of these references are cited herein and others are helpful to understand the state of the art. They are included herein for clarity and are considered incorporated herein in their entirety.
1. Geethanjali, P. Myoelectric Control of Prosthetic Hands: State-of-the-Art Review. MDER 2016, 9, 247-255.
2. Bishay, P.; Aguilar, C.; Amirbekyan, A.; Vartanian, K.; Arjon-Ramirez, M.; Pucio, D. Design of a Lightweight Shape Memory Alloy Stroke-Amplification and Locking System in a Transradial Prosthetic Arm. In Proceedings of the ASME 2021 Conference on Smart Materials, Adaptive Structures and Intelligent Systems, American Society of Mechanical Engineers, Virtual, 14 Sep. 2021; p. V001T05A015.
3. Hargrove, L.; Englehart, K.; Hudgins, B. A Training Strategy to Reduce Classification Degradation Due to Electrode Displacements in Pattern Recognition Based Myoelectric Control. Biomed. Signal Process. Control. 2008, 3, 175-180.
4. Young, A. J.; Hargrove, L. J.; Kuiken, T. A. The Effects of Electrode Size and Orientation on the Sensitivity of Myoelectric Pattern Recognition Systems to Electrode Shift. IEEE Trans. Biomed. Eng. 2011, 58, 2537-2544.
5. Stango, A.; Negro, F.; Farina, D. Spatial Correlation of High Density EMG Signals Provides Features Robust to Electrode Number and Shift in Pattern Recognition for Myocontrol. IEEE Trans. Neural Syst. Rehabil. Eng. 2015, 23, 189-198.
6. Jiang, N.; Dosen, S.; Muller, K.-R.; Farina, D. Myoelectric Control of Artificial Limbs—Is There a Need to Change Focus? [In the Spotlight]. IEEE Signal Process. Mag. 2012, 29, 150-152.
7. Beyrouthy, T.; Al Kork, S. K.; Korbane, J. A.; Abdulmonem, A. EEG Mind Controlled Smart Prosthetic Arm. In Proceedings of the 2016 IEEE International Conference on Emerging Technologies and Innovative Business Practices for the Transformation of Societies (EmergiTech), IEEE, Balaclava, Mauritius, 3-6 Aug. 2016; pp. 404-409.
8. Bright, D.; Nair, A.; Salvekar, D.; Bhisikar, S. EEG-Based Brain Controlled Prosthetic Arm. In Proceedings of the 2016 Conference on Advances in Signal Processing (CASP), Pune, India, 9-11 Jun. 2016; pp. 479-483.
9. Bishay, P. L.; Fontana, J.; Raquipiso, B.; Rodriguez, J.; Borreta, M. J.; Enos, B.; Gay, T.; Mauricio, K. Development of a Biomimetic Transradial Prosthetic Arm with Shape Memory Alloy Muscle Wires. Eng. Res. Express 2020, 2, 035041.
10. Pradeep, J.; Jamna, A.; Sasikumar, R. Low-Cost Voice-Controlled Prosthetic Arm with Five Degrees of Freedom. IETE J. Res. 2021, 69, 4047-4052.
11. Hazubski, S.; Hoppe, H.; Otte, A. Electrode-Free Visual Prosthesis/Exoskeleton Control Using Augmented Reality Glasses in a First Proof-of-Technical-Concept Study. Sci. Rep. 2020, 10, 16279.
12. Nagaraja, V. H.; da Ponte Lopes, J.; Bergmann, J. H. M. Reimagining Prosthetic Control: A Novel Body-Powered Prosthetic System for Simultaneous Control and Actuation. Prosthesis 2022, 4, 394-413.
13. Baker, C. A.; Akhlaghi, N.; Rangwala, H.; Kosecka, J.; Sikdar, S. Real-Time, Ultrasound-Based Control of a Virtual Hand by a Trans-Radial Amputee. In Proceedings of the 2016 38th Annual International Conference of the IEEE Engineering in Medicine and Biology Society (EMBC), Orlando, FL, USA, 16-20 Aug. 2016; pp. 3219-3222.
14. Engdahl, S.; Dhawan, A.; Bashatah, A.; Diao, G.; Mukherjee, B.; Monroe, B.; Holley, R.; Sikdar, S. Classification Performance and Feature Space Characteristics in Individuals with Upper Limb Loss Using Sonomyography. IEEE J. Transl. Eng. Health Med. 2022, 10, 1-11.
15. Patwardhan, S.; Schofield, J.; Joiner, W. M.; Sikdar, S. Sonomyography Shows Feasibility as a Tool to Quantify Joint Movement at the Muscle Level. In Proceedings of the 2022 International Conference on Rehabilitation Robotics (ICORR), Rotterdam, The Netherlands, 25 Jul. 2022; pp. 1-5.
16. Nazari, V.; Zheng, Y.-P. Controlling Upper Limb Prostheses Using Sonomyography (SMG): A Review. Sensors 2023, 23, 1885.
17. Lyons, K. R.; Joshi, S. S. Real-Time Evaluation of a Myoelectric Control Method for High-Level Upper Limb Amputees Based on Homologous Leg Movements. In Proceedings of the 2016 38th Annual International Conference of the IEEE Engineering in Medicine and Biology Society (EMBC), Orlando, FL, USA, 16-20 Aug. 2016; pp. 6365-6368.
18. Lyons, K. R.; Joshi, S. S. Upper Limb Prosthesis Control for High-Level Amputees via Myoelectric Recognition of Leg Gestures. IEEE Trans. Neural Syst. Rehabil. Eng. 2018, 26, 1056-1066.
19. Lyons, K. R.; Joshi, S. S. A Case Study on Classification of FOOT Gestures via Surface Electromyography. In Proceedings of the Annual Conference of Rehabilitation Engineering and Assistive Technology Society of America (RESNA), Denver, CO, USA, 11-14 Jun. 2015; pp. 1-5.
20. Maragliulo, S.; Lopes, P. F. A.; Osorio, L. B.; De Almeida, A. T.; Tavakoli, M. Foot Gesture Recognition through Dual Channel Wearable EMG System. IEEE Sens. J. 2019, 19, 10187-10197.
21. Lee, S.; Sung, M.; Choi, Y. Wearable Fabric Sensor for Controlling Myoelectric Hand Prosthesis via Classification of Foot Postures. Smart Mater. Struct. 2020, 29, 035004.
22. Resnik, L.; Klinger, S. L.; Etter, K. The DEKA Arm: Its Features, Functionality, and Evolution during the Veterans Affairs Study to Optimize the DEKA Arm. Prosthet. Orthot. Int. 2014, 38, 492-504.
23. Resnik, L.; Klinger, S. L.; Etter, K.; Fantini, C. Controlling a Multi-Degree of Freedom Upper Limb Prosthesis Using Foot Controls: User Experience. Disabil. Rehabil. Assist. Technol. 2014, 9, 318-329.
24. ten Kate, J.; Smit, G.; Breedveld, P. 3D-Printed Upper Limb Prostheses: A Review. Disabil. Rehabil. Assist. Technol. 2017, 12, 300-314.
25. Andrés, F. J.; Pérez-González, A.; Rubert, C.; Fuentes, J.; Sospedra, B. Comparison of Grasping Performance of Tendon and Linkage Transmission Systems in an Electric-Powered Low-Cost Hand Prosthesis. J. Mech. Robot. 2019, 11, 011018.
26. Bajaj, N. M.; Spiers, A. J.; Dollar, A. M. State of the Art in Artificial Wrists: A Review of Prosthetic and Robotic Wrist Design. IEEE Trans. Robot. 2019, 35, 261-277.
27. Montagnani, F.; Controzzi, M.; Cipriani, C. Is It Finger or Wrist Dexterity That Is Missing in Current Hand Prostheses? IEEE Trans. Neural Syst. Rehabil. Eng. 2015, 23, 600-609.
28. Fan, H.; Wei, G.; Ren, L. Prosthetic and Robotic Wrists Comparing with the Intelligently Evolved Human Wrist: A Review. Robotica 2022, 40, 4169-4191.
29. Belter, J. T.; Segil, J. L.; Dollar, A. M.; Weir, R. F. Mechanical Design and Performance Specifications of Anthropomorphic Prosthetic Hands: A Review. JRRD 2013, 50, 599.

Thus, specific embodiments of foot controller systems for prosthetic arms, along with their methods of production and use have been disclosed. It should be apparent, however, to those skilled in the art that many more modifications besides those already described are possible without departing from the inventive concepts herein. The inventive subject matter, therefore, is not to be restricted except in the spirit of the disclosure herein. Moreover, in interpreting the specification, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

TABLE 1

| | |
|---|---|
| | Resulting Prosthetic |
| Foot Control Action | Hand Reaction |

Wrist Control

| | | |
|---|---|---|
| 1 | Foot dorsiflexion | Wrist extension |
| 2 | Foot plantarflexion | Wrist flexion |
| 3 | Foot inversion | Forearm supination |
| 4 | Foot eversion | Forearm pronation |

Finger Actuation - Approach 1

| | | |
|---|---|---|
| 1 | Big toe single-click | Relax all fingers |
| 2 | Big toe double-click | Tripod |
| 3 | Big toe long-click | Power |
| 4 | Lesser toes single-click | Point |
| 5 | Big toe then lesser toes single-clicks | Pinch |

Finger Actuation - Approach 2

| | | |
|---|---|---|
| 1 | Big toe long-click | Close fingers of the current |
| | | grip/gesture gradually |
| 2 | Lesser toes long-click | Relax all fingers gradually |
| 3 | Big toe & lesser toes single-click | Switch grip/gesture |

TABLE 2

| | | |
|---|---|---|
| Item Name | Manufacturer | Qt |

Infinity Arm

| | | | |
|---|---|---|---|
| 1 | ESP-WROOM-32 - ESP-32S | ESPRESSIF | 1 |
| 2 | ESP32S Breakout Board | AIDEEEPEN | 1 |
| 4 | KST X06 Servo | KST Servos | 4 |
| 5 | 35 KG Coreless Digital Servo | ANNIMOS | 1 |
| 6 | 35 KG Dual Bearing Coreless | ANNIMOS | 1 |
| | Digital Servo | | |
| 7 | 2000 mAh 7.4 V Li-ion Battery | URGENEX | 1 |
| 8 | 25 T Aluminum Servo Horn | uxcell | 4 |
| 9 | Braided Fishing Line | | 1 |
| 10 | Elastic Band String | Beadsmith ® Fablastic ™ | 1 |
| 11 | 30 AWG Wire | Fermerry | 1 |

Sensor Controller Unit (SCU)

| | | | |
|---|---|---|---|
| 1 | Seed XIAO nRF52840 Sense | Seeed Studio | 1 |
| 2 | 3.7 V 400 mAh Lipo | YDL | 1 |
| 4 | 28 AWG Wire | BNTCHGO | 1 |
| 5 | SK-12D07 Slide Switch | Fielect | 1 |
| 6 | USBC Female Plug | ANMBEST | 1 |

Foot Insole Controller

| | | | |
|---|---|---|---|
| 2 | Push Buttons | QTEATAK | 2 |
| 3 | 28 AWG Wire | BNTCHGO | 1 |
| 4 | Felt | Creatology ™ | 1 |
| 5 | USBC Male Plug | ANMBEST | 1 |

1. A prosthetic arm controller system, comprising:
a first controller unit that is placed or located inside a shoe or a sock of a user, wherein the first controller unit comprises at least two interactive buttons that the user can engage, and
a second controller unit that is removably clipped or otherwise removably attached to the side or top of the shoe or the sock, wherein the second controller unit comprises at least one microcontroller with at least one integrated gyroscope, at least one accelerometer, and an onboard wireless protocol antenna to communicate at least one command to a prosthetic arm from the first controller unit or the second controller unit, wherein the at least one command comprises at least one grip command, at least one rotate command, at least one bend command, or a combination thereof.

2. The prosthetic arm controller system of claim 1, wherein the onboard wireless protocol antenna comprises a Bluetooth antenna.

3. The prosthetic arm controller system of claim 1, wherein the first controller unit comprises at least three layers.

4. The prosthetic arm controller system of claim 1, wherein the at least three layers comprises a bottom layer, a middle layer, and a top layer.

5. The prosthetic arm controller system of claim 4, wherein the bottom layer comprises thermoplastic polyurethane, the at least two interactive buttons, and at least one wire channel.

6. The prosthetic arm controller system of claim 4, wherein the middle layer comprises a silicone sheet and at least one paddle.

7. The prosthetic arm controller system of claim 4, wherein the top layer comprises fabric.

8. The prosthetic arm controller system of claim 1, wherein the second controller unit additionally comprises a rechargeable battery and an on/off switch.

9. The prosthetic arm controller system of claim 1, wherein the at least one bend command and the at least one rotate command operate to move a wrist of the prosthetic arm.

10. The prosthetic arm controller system of claim 1, wherein the at least one grip command operates to move at least one finger of the prosthetic arm.

11. The prosthetic arm controller system of claim 1, wherein the user engages the at least two interactive buttons by pressing on each button with a toe of the user.

12. The prosthetic arm controller system of claim 11, wherein the at least two interactive buttons additionally comprise at least one paddle that is located between each interactive button and the toe of the user.

13. A prosthetic arm controller system, comprising:
a first controller unit that is incorporated in a foot sleeve, wherein the first controller unit comprises at least two interactive buttons that a user can engage,
a second controller unit that is removably clipped or otherwise removably attached to the side or top of the shoe or the sock, wherein the second controller unit comprises at least one microcontroller with at least one integrated gyroscope, at least one accelerometer, and an onboard wireless protocol antenna to communicate at least one command to a prosthetic arm from the first controller unit or the second controller unit, wherein the at least one command comprises at least one grip command, at least one rotate command, at least one bend command, or a combination thereof.

14. The prosthetic arm controller system of claim 13, wherein the onboard wireless protocol antenna comprises a Bluetooth antenna.

15. The prosthetic arm controller system of claim 13, wherein the at least one bend command and the at least one rotate command operate to move a wrist of the prosthetic arm.

16. The prosthetic arm controller system of claim 13, wherein the at least one grip command operates to move at least one finger of the prosthetic arm.

17. The prosthetic arm controller system of claim 13, wherein the user engages the at least two interactive buttons by pressing on each button with a toe of the user.

18. The prosthetic arm controller system of claim 17, wherein the at least two interactive buttons additionally comprise at least one paddle that is located between each interactive button and the toe of the user.

19. The prosthetic arm controller system of claim 13, wherein the second controller unit additionally comprises a rechargeable battery and an on/off switch.
